(19) United States
(12) Patent Application Publication
(10) Pub. No.: US 2017/0340275 A1
(43) Pub. Date: Nov. 30, 2017

(54) WEARABLE DEVICE

(71) Applicant: SEIKO EPSON CORPORATION, Tokyo (JP)

(72) Inventor: Keitaro HASHIZUME, Shiojiri-shi (JP)

(73) Assignee: SEIKO EPSON CORPORATION, Tokyo (JP)

(21) Appl. No.: 15/591,612

(22) Filed: May 10, 2017

(30) Foreign Application Priority Data
May 31, 2016 (JP) 2016-108270

(51) Int. Cl. A61B 5/00; A44C 5/24; A61B 5/01; A61B 5/024

(52) U.S. Cl. CPC A61B 5/681; A61B 5/7445; A61B 5/02438; A44C 5/246; A61B 5/01; A61B 5/02416

(57) ABSTRACT

A biological information measuring device includes: a case unit; a first band that is a member connected to a first end portion of the case unit; and a second band that is a member connected to a second end portion of the case unit at the side opposite to the first end portion. When a direction from the first band toward the second band is Y axis, a direction orthogonal to the Y axis in a plan view seen from a thickness direction of the case unit is X axis, and when a width of the first end portion in the X axis direction is a and a width of the second end portion in the X axis direction is b, the case unit satisfies a relation a<b.

### CROSS-REFERENCES TO RELATED APPLICATIONS

This application claims priority to Japanese Patent Application No. 2016-108270, filed May 31, 2016, the entirety of which is herein incorporated by reference.

### BACKGROUND

### 1. Technical Field

The present invention relates to a wearable device used by being mounted on a subject.

### 2. Related Art

In the related art, a measuring device that is mounted on a wrist of a user and measures a status of a living body such as a pulse, and an electronic device such as a wristwatch having function of measuring the biological information, is known as a wearable device that functions by being mounted on a body, like clothes. For example, in JP-A-2005-204804, a wristwatch type biological information measuring device including a device main body performing the measurement and a band portion attached to the device main body, is disclosed.

In the biological information measuring device disclosed in JP-A-2005-204804, the device main body includes a case unit including a biological information measuring unit that measures biological information, and a band portion includes a first band that is connected to a first end portion of the case unit and fixed to a body (for example, a wrist) of a user and a second band that is connected to a second end portion of the case unit opposite to the first end portion of the case unit and fixed to the body of the user. In the band portion, a plurality of mounting holes are formed on the first band in the longitudinal direction, and at the other end portion opposite to an end portion connected to the second band of the case unit, a buckle having a taking rod is provided. By inserting an appropriate mounting hole on the first band into the taking rod of the buckle on the second band and fixing the band on the wrist, and then, by adjusting a tightening degree of the band portion to the wrist, it is possible to mount the device main body on the user.

Along with the recent rising awareness of health, a biological information measuring device as a wearable device becomes to be mounted for a long time or for a long period and is increasingly used for monitoring the biological information during the daily life or at the time of physical exercise. Therefore, in the biological information measuring device, demands for a miniaturization and weight reduction, an improvement in mountability and a fitness feeling are grown such that the device can endure continuous use for a long time, and it is required to reduce a visual oppression feeling as much as possible when the user sees the measuring device main body.

In addition, when continuously measuring the biological information during the physical exercise such as running for a predetermined period, it is required that the biological information measuring device includes a display unit having an excellent visibility such that the user can check a measured value of the biological information and degrees of a strength of the physical exercise and a load to the body calculated based on the measured value at any arbitrary timing.

However, in the biological information measuring device disclosed in JP-A-2005-204804, in a plan view seen from the direction facing the display unit (a dial in a timepiece), the device main body portion (the case unit) is visually recognized as relatively large in a case of a circular or a rectangular case unit. Therefore, there is a problem in that a wearer feels the visual oppression feeling, and thus, it is difficult to continuously measure the biological information of the measurement target for a long time or for a long period.

In addition, in a case where the display unit is included in the device main body portion (the case unit) that displays the measured value of the biological information and the degrees of the strength of the physical exercise and the load to the body calculated based on the measured value, the above-described problem of “the wearer feels the visual oppression feeling” can be solved. However, for example, when the size of a surface on the case unit on which the display unit is arranged is reduced, a display region on the display unit is reduced, and thus, there is a problem in that the displayed biological information and the information based thereon becomes hard to be visually recognized.

### SUMMARY

An advantage of some aspects of the invention is to solve at least a part of the problems described above, and the invention can be implemented as the following forms or application examples.

### Application Example 1

A wearable device that according to this application example includes: a case unit; a first band that is a member connected to a first end portion of the case unit for mounting the case unit on a body of a user; and a second band that is a member connected to a second end portion at the side opposite to the first end portion of the case unit for mounting the case unit on the body of the user. When a direction from the first band toward the second band is Y axis, a direction orthogonal to the Y axis in a plan view seen from a thickness direction of the case unit is X axis, and a direction orthogonal to the X axis and Y axis is Z axis, and when a width of the first end portion in the X axis direction is a and a width of the second end portion in the X axis direction is b, the case unit satisfies a relation a<b.

According to this application Example, when the user faces the case unit in a natural posture, and in a case where the wearable device is mounted such that the first end portion is positioned in front of the user (close to the line of sight), the width b of the second end portion at the back of the line of sight in the X axis direction is greater than the width a of the first end portion of the case unit in the X axis direction. Therefore, due to a perspective illusion effect, the width of the second end portion side of the case unit is visually recognized smaller than the actual width, and thus, an effect that it is difficult to give a wearer a visual oppression feeling can be obtained. Therefore, it is possible to provide the wearable device that can be continuously used for a long time while suppressing the visual oppression feeling.

In addition, due to a illusion effect described above, the width of the second end portion side of the case unit is visually recognized smaller than the actual width. Therefore, in a case where the display unit is arranged on the case unit, by setting the width b of the second end portion to be as wide as possible and arranging at least a part of display unit arranged on the surface of the case unit at the second end portion side. Therefore, the display region on the display unit can be comparatively wide. Therefore, it is possible to provide a wearable device that includes the display unit having an excellent visibility while suppressing the visual oppression feeling felt by the wearer due to the size of the case unit.

### Application Example 2

In the wearable device according to the application example, a ratio between a and b may be a:b=1:(1.1 to 1.4).

The inventors found that, if the width b is equal to or greater than 1.1 times of the width a, the user care feel the change due to the fact that the width b is greater than the width a compared to the case where the width b is the same as the width a, or if the width b is suppressed to be within 1.4 times of the width a, the user can avoid the remarkable feeling of the visual oppression feeling in which the impression received from second end portion having the width b is stronger than the impression received from the first end portion having the width a. Therefore, according to this application example, the size of the case unit can be increased while suppressing the visual oppression feeling, and thus, the display unit that has excellent visibility and can efficiently arrange the components in the case unit can be provided. Therefore, is possible to provide the wearable device that can be continuously used for long time with high functions.

### Application Example 3

In the wearable device according to the application example, when a width of the first end portion in the Z axis direction is c and a width of the second end portion in the Z axis direction is d, the relation may be c<d.

According to this application example, when the user faces the case unit, in a case where the wearable device is mounted such that the first end portion is positioned at the front side (close to the line of sight) of the wearer (user), the thickness of the case unit at the backside of the wearer (second end portion side) is greater than the thickness at the front side (first end portion side) of the wearer. That is, the surface facing the wearer of the case unit (the surface on which the first end portion and the second end portion form two opposing sides) has a rising slope such that the second end portion side which is the back side of the wearer becomes close to the wearer's line of sight. In this way, the inventors found that it is possible to obtain an effect that the display on the display unit can be visually recognized easily by, for example, arranging the display unit on the above-described slope, the backside (the second end portion side) of the display unit with respect to the wearer becomes close to the eyes of the wearer.

In addition, since the internal space of the case unit can be widened from the first end portion side close to the wearer's line of sight toward the second end portion side, the relatively large components can be arranged in the wide space at the second end portion side.

### Application Example 4

In the wearable device according to the application example, a ratio between c and d may be c:d=1:(1.2 to 4.0).

The inventors found that, if the width d is equal to or greater than 1.2 times of the width c, the user can feel the change due to the fact that the width d is greater than the width c compared to the case where the width c is the same as the width d, or if the width d is suppressed to be equal to or less than 4.0 times of the width c, the user can avoid a remarkable feeling of the visual oppression feeling in which the impression received from second end portion side having the width d is stronger than the impression received from the first end portion side having the width c. Therefore, according to this application example, in a case of increasing the size of the case unit while suppressing the visual oppression feeling and arranging the display unit in the case unit, it is possible to provide the wearable device in which the display on the display unit can be visually recognized easily.

### Application Example 5

In the wearable device according to the application example, a middle portion having a width b′ in the X axis direction which is different from at least one of the a and the b may be included between the first end portion and the second end portion in a plan view seen from the Z axis direction.

According to this application example, it is possible to obtain an effect of improving the freedom of design in the shape of case unit according to the invention in which the width b of the second end portion is greater than the width a of the first end portion while making it difficult to give the wearer the visual oppression feeling due to the perspective illusion effect.

### Application Example 6

In the wearable device according to the application example, the relation between the a and the b′ may be b′>a.

According to this application example, even when the width b′ of the middle portion is greater than the width a of the first end portion, the inventors found that it is possible to make it difficult to give the wearer the visual oppression feeling due to the perspective illusion effect.

### Application Example 7

In the wearable device according to the application example, the relation between the b and the b′ may be a≅b′ and b′≧b.

According to this application example, it is possible to provide the wearable device (case unit) in which it is difficult to give the wearer the visual oppression feeling while securing the sufficient size from the middle portion to the second end portion.

### Application Example 8

In the wearable device according to the application example, when a width of the middle portion in the Z axis direction in a plan view seen from the X axis direction is d′, the relation may be d′>c.

According to this application example, in the thickness of the case unit, the thickness of the middle portion positioned at the backside (second end portion side) of the wearer is greater than the thickness at the front side (first end portion side) of the wearer, and thus, the case unit has a slope rising to the wearer. Therefore, for example, in the configuration of arranging the display unit on the slope, it is possible to obtain an effect that the display on the display unit can be visually recognized easily.

In addition, since the internal space of the case unit can be widened from the first end portion side close to the wearer's line of sight toward the middle portion side, the relatively large components can be arranged in, for example, the wide space at the middle portion side.

### Application Example 9

In the wearable device according to the application example, when the width of the middle portion in the Z axis direction in a plan view seen from the X axis direction is d′, the relation may be d′≧d.

According to this application example, the display unit with a good visibility is arranged in the region from the middle portion to the second end portion, and the relatively large components or plural components are accommodated in the relatively larger space formed inside of the case unit from the middle portion to the second end portion while suppressing the increase of the thickness from the middle portion to the second end portion of the case unit. Therefore, it is possible to improve the function of the wearable device.

### Application Example 10

In the wearable device according to the application example, the case unit may include a biological information measuring unit that measures biological information of the user.

According to this application example, since the case unit in which it s difficult to give the wearer the visual oppression feeling while achieving the relatively large size is included, it is possible to provide the biological information measuring device as the wearable device that can be mounted for a long time or for a long period, and can monitor the biological information during daily life and physical exercise.

In addition, since the relatively large display unit can be arranged while reducing the visual oppression feeling at the time of being mounted, it is possible to obtain an effect that the measured value of the biological information or the degrees of the strength of the physical exercise and the load to the body calculated based on the measured value displayed on the display unit during, for example, running or physical exercise, can be visually recognized easily.

### Application Example 11

In the wearable device according to the application example, a battery may be included in the case unit, and a distance from the second end portion to the battery may be shorter than a distance from the first end portion to the battery in a cross sectional view seen from the X axis direction.

According to this application example, in the wearable device, it is possible to space-efficiently arrange the battery which is a relatively large component in the relatively large space secured at the second end portion side in the case unit.

### Application Example 12

In the wearable device according to the application example, an operation unit arranged on the case unit at the first end portion side in a plan view seen from the Z axis direction may be included.

According to this application example, since the operation unit is arranged in a thin region at the first end portion side of the case unit, for example, it is possible to configure the wearable device having a simple structure in which the operation unit such as the operation switch or the operation button is mounted on the circuit board arranged on the inner bottom portion of the case unit, and the operation is performed by pressing the operation unit directly or indirectly.

### Application Example 13

In the wearable device according to the application example, the case unit may include a first display unit on which a plurality of strip-shaped display units with the X axis direction as the longitudinal direction are arranged from the first end portion side toward the second end portion side.

According to this application example, it is possible to display the measured value by the wearable device, for example, the measured value of the biological information and the degrees of the strength of the physical exercise and the load to the body calculated based on the measured value in an easy-to-understand manner using the number of strips displayed by the plurality of strip-shaped display units included in the first display unit. Particularly, the lengths of each of the plurality of strip-shaped display units arranged in the X axis direction are set and arranged so as to become longer from the first end portion side toward the second end portion side according to the widths of the case unit of which the width in the X axis direction becomes larger from the first end portion toward the second end portion side. Therefore, it is possible to make the wearer visually recognize the value or degree of the displayed information with emphasizing the values or degrees.

### Application Example 14

In the wearable device according to the application example, widths of the strip-shaped display units in the Y axis direction may become larger from the first end portion side toward the second end portion side.

According to this application example, it is possible to make the wearer visually recognize the value or degree of the displayed information with further emphasizing the values or degrees.

### Application Example 15

In the wearable device according to the application example described above, a second display unit that displays an image may be arranged in a region between the strip-shaped display units adjacent to each other.

According to this application example, the information can be displayed by the image such as the characters or the numbers on the second display units, it is possible to notify the wearer of various information items in an easy-to-understand manner.

### BRIEF DESCRIPTION OF THE DRAWINGS

The invention will be described with reference to the accompanying drawings, wherein like numbers reference like elements.

FIG. 1 is an explanatory diagram illustrating a mounted state of a biological information measuring device as a wearable device in Embodiment 1 that is mounted on a living body.

FIG. 2 is a perspective view of an open state of the biological information measuring device in Embodiment 1 that is removed from the living body.

FIG. 3 is a plan view illustrating a schematic configuration of a case unit of the biological information measuring device in Embodiment 1.

FIG. 4 is a cross sectional view of the schematic configuration of the biological information measuring device in Embodiment 1 taken along the line A-A in FIG. 2.

FIG. 5 is a table illustrating results of evaluating the presence or absence and degrees of the sense of oppression felt by a user for each ratio between a width a of a first end portion in the X axis direction and a width b of a second end portion in the X axis direction on the case unit of the biological information measuring device.

FIG. 6 is a table illustrating results of evaluating the presence or absence and degrees of the sense of oppression felt by the user for each ratio between a width c of the first end portion in the Z axis direction and a width d of the second end portion in the Z axis direction on the case unit of the biological information measuring device.

FIG. 7 is a plan view illustrating a schematic configuration of a case unit of a biological information measuring device in Embodiment 2.

FIG. 8 is a cross sectional view of the schematic configuration of the biological information measuring device in Embodiment 2 taken along the same line as in FIG. 4.

FIG. 9 is a plan view illustrating a schematic configuration of a display unit in the biological information measuring device in Modification Example 1.

FIG. 10 is a plan view illustrating a schematic configuration of a display unit in the biological information measuring device in Modification Example 2.

FIG. 11 is a plan view illustrating a schematic configuration of a display unit in the biological information measuring device in Modification Example 3.

FIG. 12 is a perspective view illustrating a schematic configuration of a display unit in the biological information measuring device in Modification Example 4.

### DESCRIPTION OF EXEMPLARY EMBODIMENTS

Hereinafter, embodiments of the invention will be described referring to the drawings. In some cases, each drawing below is illustrated in a scale different from the actual scale in order to enlarge each layer and each member to the extent of recognizable degree.

### Embodiment 1

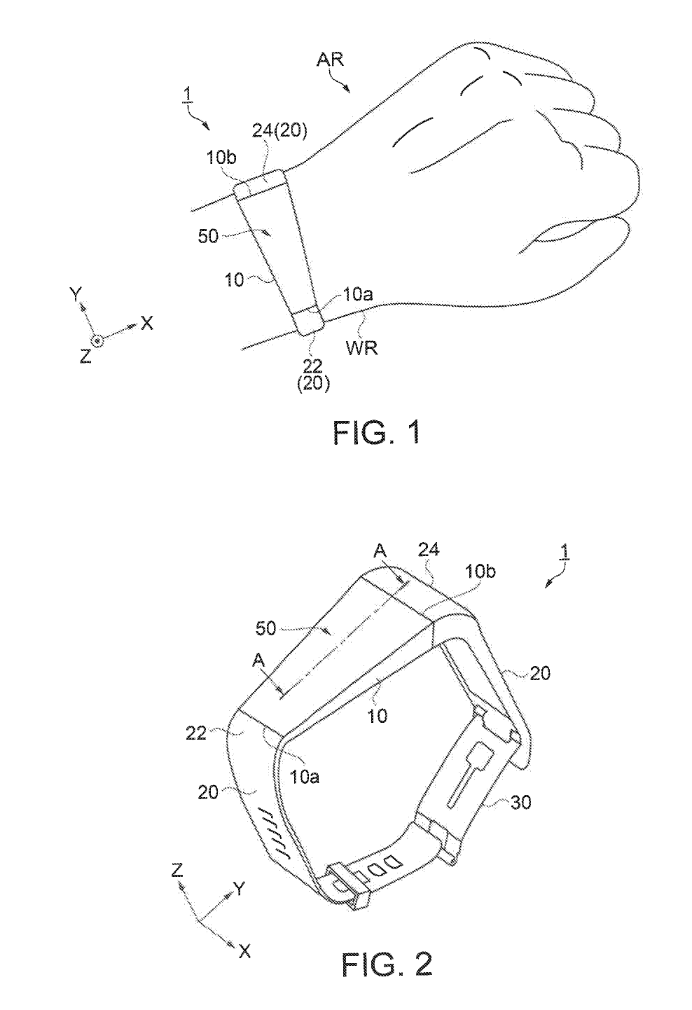

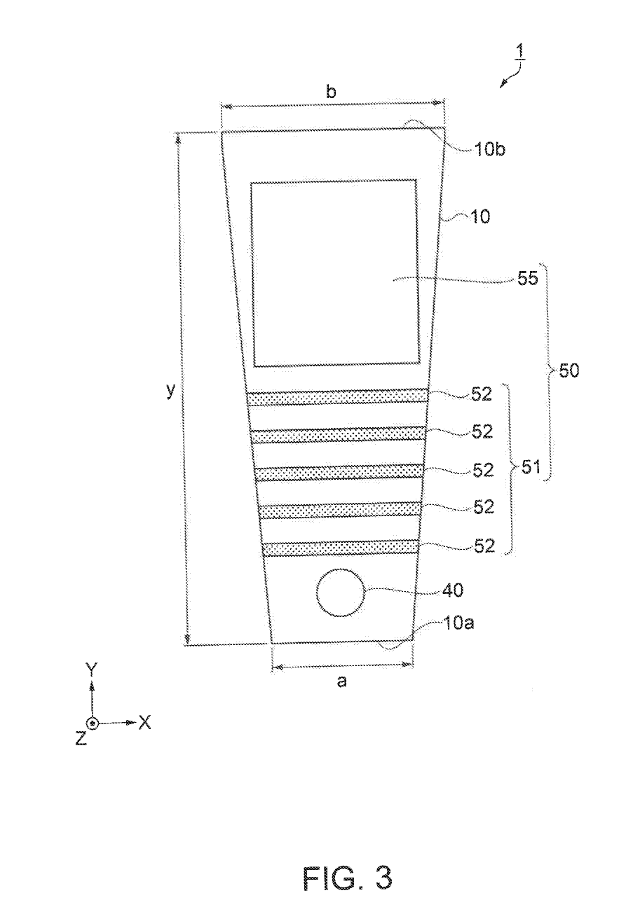

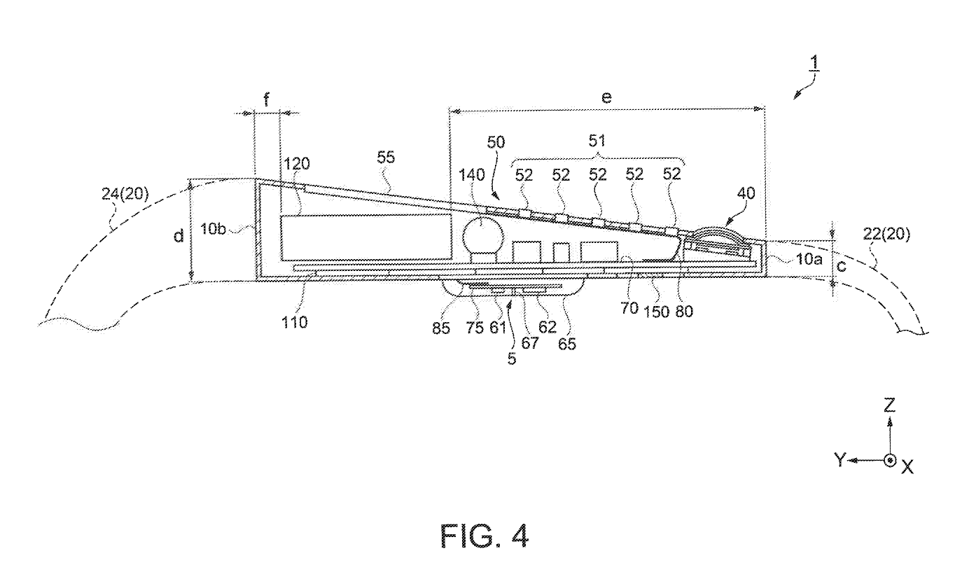

FIG. 1 is an explanatory diagram illustrating a mounted state of a biological information measuring device as a wearable device in Embodiment 1 that is mounted on a living body. FIG. 2 is a perspective view of an open state of the biological information measuring device in Embodiment 1 that is removed from the living body. FIG. 3 is a plan view illustrating a schematic configuration of a case unit of the biological information measuring device in Embodiment 1. In addition, FIG. 4 is a cross sectional view of the schematic configuration of the biological information measuring device in Embodiment 1 taken along the line A-A in FIG. 2.

First, a schematic configuration of a biological information measuring device 1 as the wearable device in Embodiment 1 will be described.

In FIG. 1, the biological information measuring device (hereinafter, referred to as a measuring device) in Embodiment 1 is an electronic device that is mounted on a living body (for example, a human body) of which biological information is measured, and measures a pulse or other biological information items. The measuring device 1 in Embodiment 1 is mounted on a measuring part of a user (living body) like a wristwatch and used.

In this specification, a normal direction on a front surface of the measuring device 1 is assumed to be a Z axis direction with a front side in FIG. 1 as positive. Here, the front surface of the measuring device 1 means a surface on which a display unit 50 is arranged. In addition, a length direction of an arm AR crossing the Z axis direction is assumed to be an X axis direction with a distal end side with fingers as positive. A width direction of the arm AR crossing the Z axis direction and the X axis direction is assumed to a Y axis direction with a little-finger side as positive. In addition, a normal direction of a display surface of the display unit 50 described below may be the Z axis, a direction orthogonal to the Z axis and toward a second band from a first band described below or a direction toward a second end portion from a first end portion of a case unit may be the Y axis, and a direction orthogonal to the Z axis and the Y axis may be the X axis. p In addition, in this specification, the measuring device 1 seen from the normal direction on the front surface (Z axis direction) is referred to a “front view”. Alternatively, the “front view” may be a projection view seen from the Z axis direction. In addition, the measuring device 1 seen from the X axis direction is referred to as a “side view”. In addition, a living body side, that is, a side facing the wrist WR in a state in which the measuring device 1 is mounted on the wrist WR is referred to as an “inner side” or an “inner surface” a side opposite to the living body, that is, a side opposite to the side facing the wrist WR is referred to as an “outer side” or an “outer surface”. A cross section of the measuring device 1 seen from the same direction as the above-described “side view”, that is, the measuring device 1 seen from the crass section by the line A-A in FIG. 2 is referred to as a “cross sectional view”. In this specification, these “front view”, “side view”, and “cross sectional view” are important drawing directions and will be occasionally described below in detail.

As illustrated in FIG. 2, the measuring device 1 includes a case unit 10 that is a device main body, a band portion 20 including a first band 22 and a second band 24 that fix the case unit 10 to the wrist WR, and a buckle portion 30 as a connection portion that connects the first band 22 and the second band 24 of the band portion 20.

The display unit (a monitor unit (display)) 50 that displays a result of measuring the biological information or the measuring time with characters or graphs is provided at the front surface side of the case unit 10. The measuring device 1 measures the biological information in a state in which a bottom surface (detection unit) at the opposite side of the front surface of the measuring device 1 is in close contact with the wrist WR. The result of measuring and the result of evaluating the biological information based on the result of measuring are displayed on the display unit 50.

The first band 22 of the band portion 20 is connected to a first end portion 10a which is one end portion of the case unit 10, and the second band 24 is connected to a second end portion 10b which is the other end portion (end portion at the opposite side of the first end portion 10a) of the case unit 10, and the distal end sides of the first band 22 and the second band 24 are linked by the buckle portion 30. The buckle portion 30 in the present embodiment is a hinged member in which two metal plates are connected by a rotation shaft and the length becomes short when two plates overlap to be folded and becomes long when stretched out. That is, the measuring device 1 includes a case unit 10 including the detection unit that detects the biological information, the band portion 20 for fixing the case unit 10 to the living body, and the buckle portion 30 which is in connection with the band portion 20 in an annular shape and the length of which can be adjusted.

As described above, by both end portions (the end portions of the first band 22 and the second band 24) of the band portion 20 being linked by the buckle portion 30, the measuring device 1 has an annular shape in both states: the state in which the device is mounted on the wrist WR (hereinafter, referred to as a mounted state) as illustrated in FIG. 1 and a state in which the device is removed from the wrist WR (hereinafter, referred to as an open state) as illustrated in FIG. 2.

According to this configuration, when mounting the measuring device 1, in the state in which the buckle portion 30 is stretched, by folding and shortening the buckle portion 30 at the mounting position on the arm after pushing down and inserting the hand into the annular-shape large opening illustrated in FIG. 2, it is possible to mount the measuring device 1 as illustrated in FIG. 1. Particularly, by applying various creative ideas such as the optimization of the configurations and the materials of the band portion 20 and the size of the annular-shape opening portion, even if the measuring device 1 is repeatedly mounted and removed, it is possible to realize the configuration such that a detection unit (a biological information measuring unit) such as a pulse wave sensor unit 5 described below can be fixed on the detection position on the arm accurately at a substantially same pressure (pressing force).

In the present embodiment, the type of mounting the device using the band portion 20 and the folding type buckle portion 30 is described. However, a type connecting a band and a connection portion may be used, in which a buckle and a taking rod are included in one end portion side of the band portion and small holes are provided on a stitch at the other end portion side of the band portion, and then, the stitch on the other end portion side is passed through the buckle and the is inserted into the small holes to be fixed.

The plan view of the measuring device 1 illustrated in FIG. 3 is a view illustrated as the above-described “front view” of the case unit 10 of the measuring device 1, and the illustration of the band portion 20 is omitted. In the case unit 10 of the measuring device 1, when the width of the first end portion 10a in the X axis direction is “a” and the width of the second end portion 10b in the X axis direction “b” in the front view, the width b is greater than the width a (a<b). When the width between the first end portion 10a and the second end portion 10b in the Y axis direction is “y”, an appropriate ratio between the width a of the first end portion 10a and the width b of the second end portion 10b will be described below in detail.

As described above, the display unit 50 is proved on the “front surface” of the case unit 10 in the front view. The display unit 50 in the present embodiment includes: a first display unit 51 in which strip-shaped display units 52 having strip shapes with the X axis direction as the longitudinal direction are arranged at the front of the case unit 10 in the direction toward the second end portion 10b side from the first end portion 10a side; and a second display unit 55 that is arranged at the second end portion 10b of the first display unit 51 and displays characters and the like as an image.

In the strip-shaped display units 52 included in the first display unit 51, for example, light emitting elements such as light emitting diodes (LED) are used. In the present embodiment, five of a plurality of strip-shaped display units 52 having the same shape are arranged at a regular interval. The first display unit 51 can notify the user of the measured value of the biological information measured by the detection unit (the biological information measuring unit) such as the pulse wave sensor unit 5 and the degrees of the strength of the physical exercise and the load to the body calculated based on the measured value, using the number of strip-shaped display units 52 that are lit among the plurality of strip-shaped display units 52 so as to be easily understood. In addition, the strip-shaped display units 52 are configured such that the width increases along the direction toward the second end portion 10b side from the first end portion 10a side. In this configuration, the user can easily and intuitively understand the degrees of the degrees of the strength of the physical exercise and the load to the body.

In addition, for example, a liquid crystal display (LCD) can preferably used in the second display unit 55. The second display unit 55 can display the measured value of the biological information measured by the detection unit (the biological information measuring unit) such as the pulse wave sensor unit 5 in real time in numbers or the like, and can notify the user of the degrees of the strength of the physical exercise and the load to the body based on the measured value or other message information using the characters and the image. In addition, since the second display unit 55 is arranged at the second end portion 10b side of the first display unit 51, particularly the width in the X axis direction can be provided relatively wide. In this way, the image such as characters or the numbers displayed on the second display unit 55 can be displayed large. Therefore, it is possible to obtain an effect that the user can visually recognize the displayed image information easily.

In addition, an operation unit 40 such as an operation switch or an operation button that performs the switching of the operation of the measuring device 1 is provided at the first end portion 10a side of the display unit 50 on the “front surface” of the measuring device 1 in the present embodiment. Details of the operation unit 40 will be described below.

The cross sectional view of the measuring device 1 illustrated in FIG. 4 is a view illustrated as the above-described “cross sectional view” of the case unit 10 of the measuring device 1. In the case unit 10 of the measuring device 1 in FIG. 4, when the width of the first end portion 10a in the Z axis direction is “c” and the width of the second end portion 10b in the Z axis direction is “d”, the width d is greater than the width c (c<d). Therefore, an internal space formed inside of the case unit 10 is wider at the first end portion 10a side than that at the second end portion 10b side. Therefore, relatively large components can be arranged and accommodated in the space at the second end portion 10b side. In the present embodiment, a battery 120 is provided inside of the case unit 10 as the relatively large component in the wearable device such as the measuring device 1, a distance f from the second end portion 10b to the battery 120 is longer than a distance e first end portion 10a to the battery 120 in the cross sectional view seen from the X axis direction, that is, the battery 120 is arranged in the space inside of the case unit 10 at the second end portion 10b side. In addition, in the immediate vicinity of the arrangement position of the battery 120 inside of the case unit 10, relatively large components 140 other than the battery 120 are arranged.

A main circuit board 70 on which a main driving and control circuit of the biological information measuring device 1 including a driving circuit of the display unit 50 and a below-described biological information measuring unit (detection unit) are mounted is arranged at a bottom surface side (a surface side facing the “front surface” at which the display unit 50 is arranged) inside of the case unit 10. The main circuit board 70 and the display unit 50 are electrically connected by a flexible substrate (hereinafter, referred to as a flexible printed circuit (FPC) 80) at the first end portion 10a side inside of the case unit 10. The length between the main circuit board 70 and the display unit 50 at the first end portion 10a side is shorter than that at the second end portion 10b side. Therefore, the connection length of the main circuit board 70 and the display unit 50 by the FPC 80 becomes short, and it is possible to reduce the size of the component (the FPC 80). Thus, it is advantageous for miniaturization of the biological information measuring device 1.

At the outer side of the bottom surface of the case unit 10, that is, at the living body side (wrist side) of the user, the pulse wave sensor unit 5 as the biological information measuring unit that detects the pulse wave as the biological information is arranged. In the present embodiment, a sensor projection portion 65 protruding to the living body side of the user is formed on a surface at the outer side of the bottom surface of the case unit 10, and the pulse wave sensor unit 5 is mounted on a circuit board 75 for a sensor arranged in the internal space of this sensor projection portion 65. The circuit board 75 for a sensor is electrically connected to the main circuit board 70 via a relay substrate 85 such as the FPC. The sensor projection portion 65 can be a lib that is formed integrally with the case unit 10. As described above, by arranging the pulse wave sensor unit 5 on the sensor projection portion 65 protruding to the living body side of the user, a contact state of a pulse wave sensor to a body of the user can be stable, and thus, it is possible to achieve an effect of improving the detection accuracy of a pulse wave signal. In addition, in the biological information measuring device 1 in the present embodiment, the circuit board 75 for a sensor on which the pulse wave sensor unit 5 is provided in addition to the main circuit board 70 on which the main driving and control circuit and a below-described various detection units are mounted. In this way, in the biological information measuring device 1 which is required to be reduced in size, it is possible to realize the improvement of a degree of freedom of the layout of the pulse wave sensor unit 5 or various other detection units in the case unit 10.

Here, the configuration and a measurement principle of the pulse wave sensor unit 5 will be described. The pulse wave sensor unit 5 is a photoelectric pulse wave sensor that includes a light emitting element 61 (usually, LEDs are used) as a light source mounted on a surface of the circuit board 75 for sensor and the circuit board 75 for a sensor arranged in the sensor projection portion 65 at the living body side (skin of the wrist) of the user, and a light receiving element (usually, photodiodes are used). The light emitting element 61 and the light receiving element 62 in the sensor projection portion 65 are blocked by a light blocking member 67, and thus, a window portion of the skin side (at least a part of the sensor projection portion 65) of the light emitting element 61 and the light receiving element 62 has translucency. The window portion is a member having a convex lens shape and transparent resin is preferably used as the window portion. A part of the light passing through the window portion from the light emitting element 61 of the pulse wave sensor unit 5 and emitted to the skin (living body) of the user is absorbed by the blood flowing in the blood vessel under the skin, but the remaining light is reflected and comes out to the skin. The light reflected from the living body is captured by the light receiving element 62 and output to the main circuit board 70 via the relay substrate 85 as a light receiving signal. The light receiving signal from the light receiving element 62 is a signal that includes information corresponding to an amount of blood flowing through the blood vessel. The amount of blood flowing through the blood vessel is changed by a pulsation of the heart, and thus, the signal on the light receiving element 62 changes according to the heart beat. That ice, the change of the signal of the light receiving element 62 corresponds to the pulse the heart rate. By counting the number of pulses per a unit time (for example, per 10 seconds), the heart rate which is the number of heart beats per one minute can be obtained.

In addition to the pulse wave sensor unit 5 described above, by mounting various sensors or electronic components on the biological information measuring device 1, it is possible to provide the measuring device 1 having more versatile functions. For example, a vibration motor 110 is mounted at the pulse wave sensor unit 5 side of the main circuit board the measuring device 1 in the present embodiment, that is, on the surface (lower surface) at the living body side of the user. The vibration motor 110 transfers the information such as the determination result of the state of the user which is based on the result of measuring the biological information measured bar the biological information measuring device 1 to the user using the vibration. As in the present embodiment, by arranging the vibration motor 110 on the surface at the living body side same as the position where the pulse wave sensor unit 5 of the main circuit board 70 is arranged, it is possible for the user to easily feel the information notification by the vibration of the vibration motor 110.

In addition, the measuring device 1 having the more versatile functions can be configured by mounting a temperature sensor that detects a temperature of the user (living body), that is, a body temperature, a body movement sensor such as an acceleration sensor or an angular velocity sensor, or a GPS unit. The temperature sensor can also measure an environmental temperature.

In addition, a charging terminal portion 150 for charging the battery 120 is provided on one surface of the main circuit board 70.

In addition, as described above, the operation unit 40 is provided at the first end portion 10a side of the display unit 50 in front of the case unit 10. In FIG. 4, a membrane switch type operation unit 40 is illustrated, which is configured to include a surface sheet having a projection portion arranged in front of the case unit 10, an upper contact point, a lower contact point, and a spacer interposed between the upper contact point and the lower contact point in order for making the upper contact point and the lower contact point not to come in contact with each other in an ordinary time, and is configured such that the upper contact point and the lower contact point come in contact with each other when the projection portion of the surface sheet is pressed by a finger. As described above, by the operation unit 40 being provided in a region at the first end portion 10a side in front of the case unit 10, the operation unit 40 is arranged in a region where the thickness of the case unit 10 at the first end portion 10a side on the “front surface” of the case unit 10 is thin (where the width c in the Z axis direction is narrow). In this way, it is possible to configure the biological information measuring device (wearable device) 1 having a simple structure in which the operation unit 40 such as the operation switch or the operation button is mounted on the main circuit board 70 arranged on the inner bottom portion of the case unit 10, and predetermined operations of the measuring device 1 are performed by operating the operation unit 40 directly (or indirectly). In addition, in a case of attempting the electrical connection of the operation unit 40 and the main circuit board 70, since the distance between the operation unit 40 and the main circuit board 70 is shorter than that in the second end portion 10b side, for example, there is an advantage of reducing the connection length using the connection wiring material such as FPC.

The inventors evaluated the presence or absence and the degree of the sense of oppression felt by the user for each ratio b/a between the width a of the first end portion 10a in the X axis direction and the width b of the second end portion 10b in the X axis direction (refer to FIG. 3) on the “front surface” of the case unit 10 described above by performing a keen verification and confirmation, and found a preferable configuration of the ratio b/a. Specifically, it was found that the ratio a:b between the width a of the first end portion 10a in the X axis direction and the width b of the second end portion 10b in the X axis direction is a:b=1:(1.1 to 1.4) is preferable.

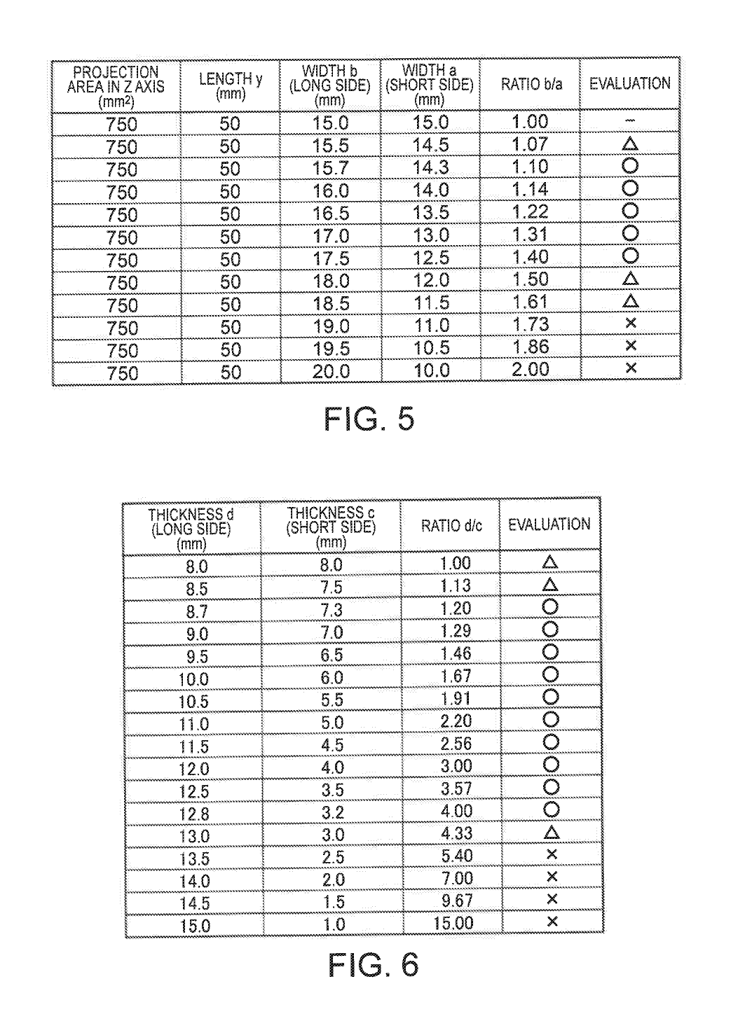

The result of evaluating the presence or absence and the degree of the sense of oppression felt by the user for each ratio between the width a of the first end portion 10a in the X axis direction and the width b of the second end portion 10b in the X axis direction in the case unit 10 of the biological information measuring device 1, is illustrated in a table in FIG. 5. In the table in FIG. 5, by mounting the samples of the case units created by combining a plurality of widths (short sides) a of the first end portion 10a and the widths (long sides) b of the second end portion 10b on the wrist of the subject via the band portion in a level by level, the presence or absence and the degrees of the sense of oppression felt by the user were evaluated. The evaluation was made while the width y between the first end portion 10a and the second end portion 10b in the Y axis direction (refer to FIG. 3) on the “front surface” of the case unit 10 is fixed to be 50 mm.

In the table in FIG. 5, a case where the change from the case where the ratio between the width a and the width b is a:b=1:1 is felt but the sense of oppression is not felt is indicated as “preferred (O)”, a case where the change from the case of the ratio a:b=1:1 is felt and the sense of oppression is slightly felt is indicated as “yes (Δ)”, and a case where the change from the case of the ratio a:b=1:1 is felt and the sense of oppression is clearly felt is indicated as “no (X)”.

As illustrated in FIG. 5, in cases where the ratio a:b between the width a and the width b is 1:1.10, 1:1.14, 1:1.22, 1:1.31, and 1:1.40, the evaluation results “preferred (O) ” are seen. From this evaluation result, the preferable range of the ratio a:b between the width a of the first end portion 10a and the width b of the second end portion 10b of the case unit 10 can be defined as 1:(1.1 to 1.4). That is, if the width b is equal to or greater than 1.1 times of the width a, the change due to the fact that the width b is greater than the width a can be felt compared to the case where the width b is the same as the width a, and if the width b is suppressed to be within 1.4 times of the width a, it is possible to avoid a remarkable feeling of the visual oppression feeling in which the impression received from second end portion 10b having the width b is stronger than the impression received from the first end portion 10a having the width a. Therefore, by making the ratio a:b between the width a and the width b on the “front surface” of the case unit 10 be 1:(1.1 to 1.4), it is possible to increase the size of the case unit 10 while suppressing the visual oppression feeling, and thus, it is possible to provide the biological information measuring device 1 as a wearable device that includes the display unit 50 performing large display and having excellent visibility.

In addition, the inventors evaluated the presence or absence and the degree of the sense of oppression felt by the user for each ratio d/c between the width c of the first end portion 10a in the Z axis direction and the width d of the second end portion 10b in the Z axis direction (refer to FIG. 4) in “side view” or the cross sectional view” of the case unit 10 described above, and found a preferable configuration of the ratio d/c. Specifically, it was found that the ratio c:d between the width c of the first end portion 10a in the Z axis direction and the width d of the second end portion 10b in the Z axis direction is c:d 1:(1.2 to 4.0) preferable.

The result of evaluating the presence or absence and the degree of the sense of oppression felt by the user for each ration d/c between the width a of the first end portion 10a in the Z axis direction and the width b of the second end portion 10b in the Z axis direction in the case unit 10 of the biological information measuring device 1, is illustrated in a table in FIG. 6. In the table in FIG. 6, by mounting the samples of the case units created by combining a plurality of widths (thicknesses) a of the first end portion 10a and the widths (thicknesses) b of the second end portion 10b on the wrist of the user via the band portion in a level by level, the presence or absence and the degrees of the sense of oppression felt by the user were evaluated. Although not illustrated in the drawing, the evaluation was performed with the width y in the Y axis direction between the first end portion 10a and the second end portion 10b of the case unit as the same 50 mm similarly to the evaluation in the table in FIG. 5.

In the table in FIG. 6, a case where the change from the case where the ratio between the width c and the width d is c:d=1:1 is felt but the sense of oppression is not felt is indicated as “preferred (O)”, a case where the change from the case of the ratio c:d=1:1 is felt and the sense of oppression is slightly felt indicated as “yes (Δ)”, and a case where the change from the case of the ratio c:d=1:1 is felt and the sense of oppression is clearly felt is indicated as “no (X)”.

As illustrated in FIG. 6, in cases where the ratio c d between the width c and the width d is 1:1.20, 1:1.29, 1:1.46, 1:1.67, and 1:1.91, 1:2.20, 1:2.56, 1:3.00, 1:3.57, and 1:4.00, the evaluation results “preferred (O)” are seen. From this evaluation result, the preferable range of the ratio c:d between the width (thickness) c of the first end portion 10a and the width (thickness) d of the second end portion 10b of the case unit 10 can be defined as 1:(1.2 to 4.0). That is, if the width d is equal to or greater than 1.2 times of the width c, the change due to the fact that the width d is greater than the width c can be felt compared to the case where the width d is the same as the width c, and if the width d is suppressed to be within 4 times of the width c, it is possible to avoid a remarkable feeling of the visual oppression feeling in which the impression received from second end portion 10b having the width d is stronger than the impression received from the first end portion 10a having the width c. In addition, in the mounted state of the measuring device 1, the “front surface” on the case unit 10 on which the display unit 50 is arranged has a rising slope such that the second end portion 10b which is at the back side of the user's eye compared to the first end portion 10a becomes close to the user's eye. Therefore, it is possible to obtain an effect that the display on the display unit 50 can be visually recognized easily.

Therefore, by making the ratio c:d between the width c and the width d in “side view” or in “cross sectional view” of the case unit 10 be 1:(1.2 to 4.0), it is possible to increase the size of the case unit 10 while suppressing the visual oppression feeling, and thus, the components can be efficiently arranged in the case unit 10. Therefore, it is possible to provide the biological information measuring device 1 as a wearable device that can be continuously used for a long time with higher function.

As described above, according to the biological information measuring device 1 in the described Embodiment 1, when the width of the first end portion 10a of the case unit 10 in the X axis direction is width a and the width of the second end portion 10b in the X axis direction is width b, the configuration is made to be a<b. In this way, when the biological information ensuring device 1 is mounted, the first end portion 10a is positioned in front side of the user (close to the line of sight), the width b of the second end portion 10b in the X axis direction at the back of the line of sight is greater than the width a of the first end portion 10a of the case unit 10 in the X axis direction. Therefore, due to a perspective illusion effect, the width b of the second end portion 10b of the case unit 10 is visually recognized smaller than the actual width, and thus, an effect that it is difficult to give a wearer a visual oppression feeling can be obtained. Therefore, it is possible to provide the biological information measuring device 1 as a wearable device that can be continuously used for a long time while suppressing the visual oppression feeling.

In addition, due to a illusion effect described above, the width b of the second end portion 10b of the case unit 10 is visually recognized smaller than the actual width. Therefore, by setting the width b of the second end portion 10b to be as wide as possible and arranging at least a part of display unit 50 arranged on the surface of the case unit 10 at the second end portion 10b side, the display region on the display unit 50 can be comparatively wide. Therefore, it is possible to provide the biological information measuring device 1 as a wearable device that includes the display unit 50 having an excellent visibility while suppressing the visual oppression feeling felt by the wearer due to the size of the case unit 10.

In addition, in the present embodiment, the relation between the width (thickness) c of the first end portion 10a of the case unit 10 in the Z axis direction and the width (thickness) d of the second end portion 10b in the Z axis direction the configuration is made to be c<d.

In this way, when the user wearing the biological information measuring device 1 faces the front surface of the case unit 10, the first end portion 10a is positioned at the front side (close to the line of sight) of the wearer (user), and thus, the thickness of the case unit 10 at the backside of the wearer (second end portion 10b side) is greater than the thickness at the front side (first end portion 10a side) of the wearer. That is, the surface facing the wearer of the case unit 10 (front surface, the surface on which the, display unit 50 is arranged) has a rising slope such that the second end portion 10b which is the back side of the wearer becomes close to the wearer's line of sight. In this way, it is possible to obtain an effect that the display on the display unit 50 can be visually recognized easily.

In addition, since the internal space of the case unit 10 can be widened from the first end portion 10a side close to the wearer's line of sight toward the second end portion 10b side, the battery 120 and other relatively large components 140 can be arranged in the relatively wide space the second end portion 10b side.

### Embodiment 2

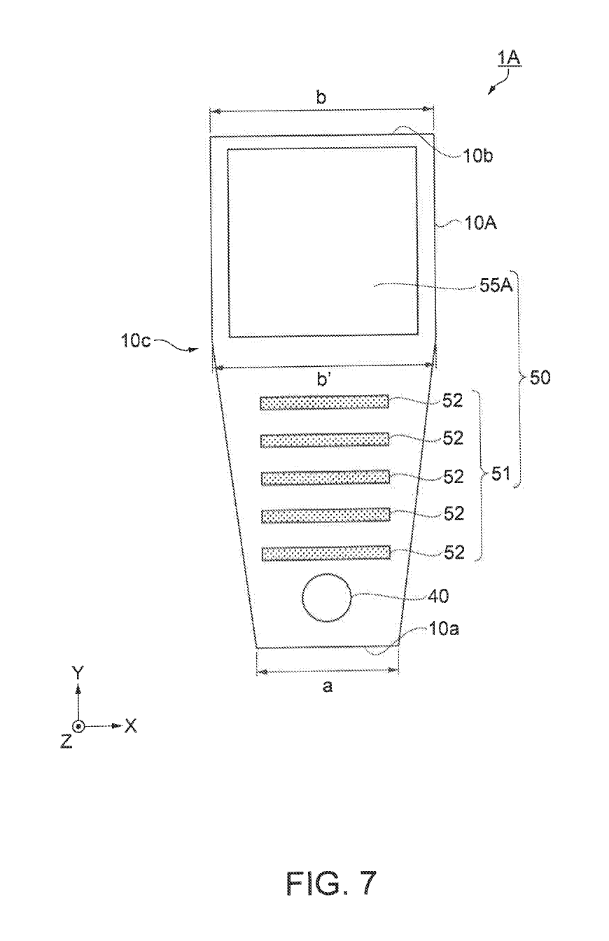

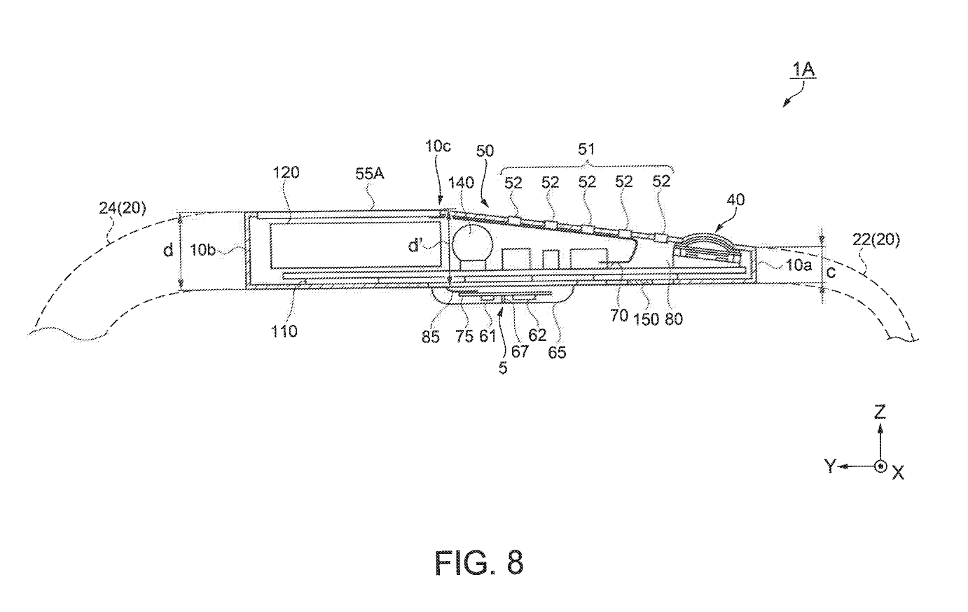

FIG. 7 is a plan view illustrating a schematic configuration of a case unit of a biological information measuring device in Embodiment 2. In addition, FIG. 8 is a cross sectional view of the schematic configuration of a case unit of the biological information measuring device in Embodiment 2 taken along the same line as ins FIG. 4.

Hereinafter, a biological information measuring device 1A as a wearable device in Embodiment 2 will be described with reference to the drawings. The same reference signs will be given to configuration portions same as those in Embodiment 1, and the descriptions thereof will not be repeated.

The measuring device 1A in Embodiment 2 illustrated in FIG. 7 is a view illustrated as the above-described “front view” of a case unit 10A, that is, in the Z axis direction, and the illustration of the band portion is omitted. One of large differences between the measuring device 1 in Embodiment 1 and the measuring device 1A in the present embodiment is a shape of the front view of the case unit 10A. In the case unit 10A of the measuring device 1A in Embodiment 2 illustrated in FIG. 7, in the front view, the width b of the second end portion 10b in the X axis direction is greater than the width a of the first end portion 10a in the X axis direction (a<b), and the case unit 10A includes a middle portion 10c having a width b′ that is a width between the first end portion 10a and the second end portion 10b in the X axis direction and is different from at least one of the width a of the first end portion 10a and the width b of the second end portion 10b. In this way, it is possible to obtain an effect of improving the degree of freedom of design in the shape of case unit 10A in which the width b of the second end portion 10b is greater than the width a of the first end portion 10a while making it difficult to give the wearer the visual oppression feeling due to the perspective illusion effect.

In the shape of the front view of the case unit 10A described above, in the present embodiment, the relation between the width a of the first end portion 10a and the width b′ of the middle portion 10c is still b′>a. In this way, even when the width b′ of the middle portion 10c is greater than the width a of the first end portion 10a, it is possible to make it difficult to give the wearer the visual oppression feeling due to the perspective illusion effect, and it is possible suppress a dead space from the middle portion 10c to the second end portion 10b compared to the case of the case unit 10 in Embodiment 1. Specifically, in a case of the configuration in which the widths of the first end portion 10a and the second end portion 10b are the same as those in the case unit 10 in Embodiment 1, it is possible to secure a larger space on the “front surface” from the middle portion 10c to the second end portion 10b, and thus, for example, it is possible to provide the second display unit 55A with a larger size than that in the second display unit 55 (refer to FIG. 3) in the case unit 10 in Embodiment 1.

Furthermore, in the case unit 10A described above, it is preferable that the relation between the width b of the second end portion 10b and the width b′ of the middle portion 10c is b′≧b, and the relation in the case unit 10A in the present embodiment is b′=b. According to this configuration, it is possible to provide the measuring device 1A (case unit 10A) in which it is difficult to give the wearer the visual oppression feeling while securing the sufficient size from the middle portion 10c to the second end portion 10b.

FIG. 8 is a view of the case unit 10A of the measuring device 1A in the present embodiment as the above-described “cross sectional view”, and similarly to the case unit 10 in Embodiment 1, the width d of the second end portion 10b is greater than the width c of the first end portion 10a in the Z axis direction (c<d), and when the width of the middle portion 10c in the Z axis direction width d′, the relation is d′>c. In this way, in the thickness of the case unit 10A, the thickness of the middle portion 10c (width d′) positioned at the backside (second end portion 10b side) of the wearer is greater than the thickness (width c) at the front side (first end portion 10a side) of the wearer, and thus, the case unit 10A has a slope rising to the wearer. Therefore, in the configuration of arranging the display unit 50 on the slope, it is possible to obtain an effect that the display on the display unit 50 can be visually recognized easily.

In addition, since the internal space of the case unit 10A can be widened from the first end portion 10a side close to the wearer's line of sight toward the middle portion 10c side, the battery 120 and other relatively large components 140 can be arranged in, for example, the wide space at the middle portion 10c side.

Furthermore, in the “cross sectional view (plan seen from the X axis direction)” of the case unit 10A in the present embodiment, it is preferable that the relation between the width d of the second end portion 10b in the Z axis direction and the width d′ of the middle portion 10c in the Z axis direction is d′≧d, and the relation in the present Embodiment is d′=d. In this way, the display unit 50 with a good visibility is arranged in the region from the middle portion 10c to the second end portion 10b, and the battery 120 and other relatively large components 140 or other plural number of components are accommodated in the relatively larger space formed inside of the case unit from the middle portion 10c to the second end portion 10b while suppressing the increase of the thickness from the middle portion 10c to the second end portion 10b of the case unit 10A and the increase of the dead space. Therefore, it is possible to improve the function of the biological information measuring device 1A as the wearable device without increasing the size.

The invention is not limited to the embodiments described above, and various changes and modification can be made added to the embodiments described above. Modification examples will be described below.

In the embodiments described above, the display unit arranged on the “front surface” of the case unit is configured to include the first display unit 51 in which a plurality of strip-shaped display units 52 having the same shapes with the X axis direction as the longitudinal direction are arranged along direction from the first end portion 10a toward the second end portion 10b, and the second display unit 55 such as LCDs displaying the characters and image and arranged at the second end portion 10b side of the first splay unit 51. However, the invention is not limited thereto.

Hereinafter, four modification examples of the display units of the biological information measuring device as the wearable device will be described with reference to the drawings. In modification examples described below, the same reference signs will be given to configuration portions same as those in Embodiment 1, and the descriptions thereof will not be repeated.

### Modification Example 1

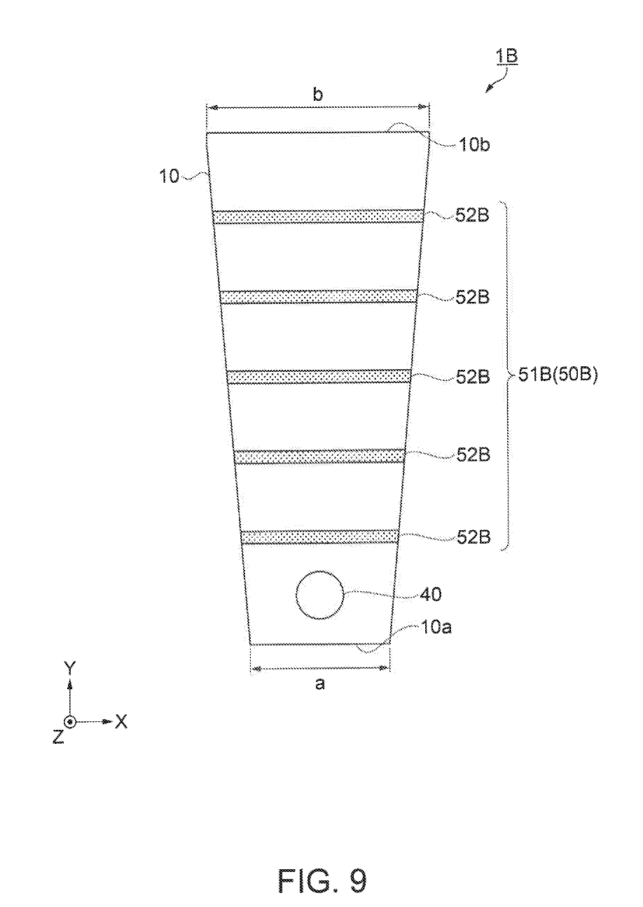

FIG. 9 is a plan view illustrating a schematic configuration of a display unit in the biological information measuring device in Modification Example 1.

In FIG. 9, a display unit 50B in a measuring device 1B in Modification Example 1 is configured to include only the first display unit 51B in which a plurality of strip-shaped display units 52B with the X axis direction as the longitudinal direction are arranged along direction from the first end portion 10a side toward the second end portion 10b side (Y axis direction). The widths of the plurality of strip-shaped display units 52B are formed to be the same in the Y axis direction, and are arranged in a regular interval in the Y axis direction. However, the widths in the X axis direction which is the longitudinal direction are formed to become longer from the first end portion 10a side to the second end portion 10b side. That is, the width of each of the plurality of strip shaped display units 52B arranged on the “front surface” of the case unit 10 case unit 10 in the X axis direction is set according to the shape of the “front surface” of the case unit 10 of which the width in the X axis direction becomes longer from the first end portion 10a toward the second end portion 10b.

Even if the display unit 50B as in the measuring device 1B in the present modification example is configured to include only the first display units 51B including the plurality of strip-shaped display units 52B, it is possible to display the measured value of the biological information measured by the measuring device 1B and the degrees of the strength of the physical exercise and the load to the body calculated based on the measured value in an easy-to-understand manner using the number of strips displayed by the plurality of strip-shaped display units 52B included in the display unit 50B (first display unit 51B). In the present modification example, the lengths of each of the plurality of strip-shaped display units 52B arranged in the X axis direction are set and arranged so as to become longer from the first end portion 10a side to the second end portion 10b side according to the widths of the case unit 10 of which the width in the X axis direction becomes longer from the first end portion 10a toward the second end portion 10b. Therefore, it is possible to make the wearer visually recognize the value or degree of the displayed information with emphasizing the values or degrees.

### Modification Example 2

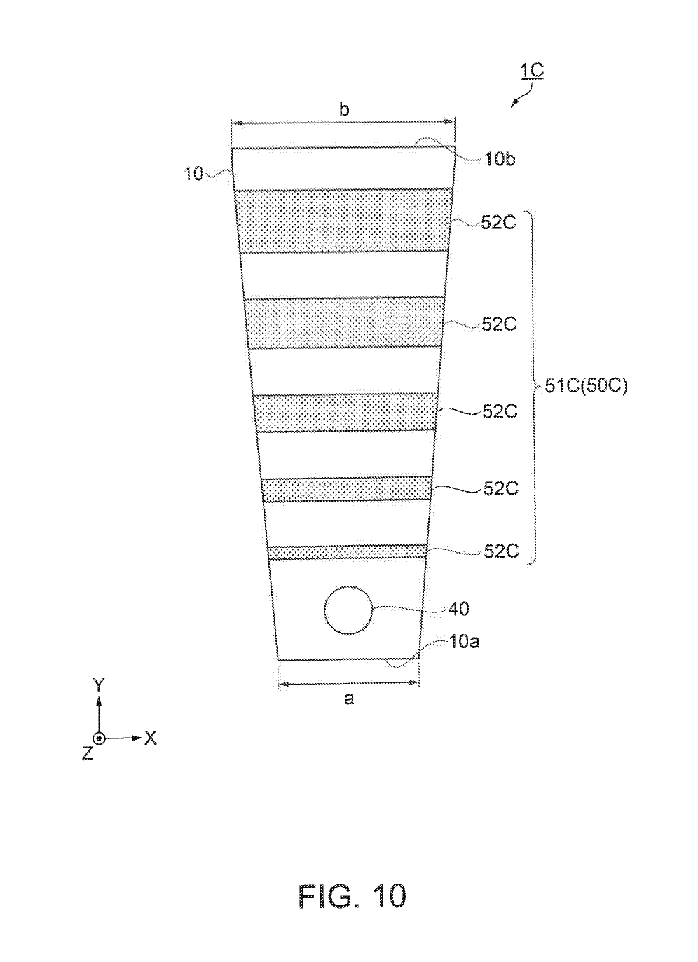

FIG. 10 is a plan view illustrating a schematic configuration of a display unit in the biological information measuring device in Modification Example 2.

In FIG. 10, similarly to Modification Example 1, the display unit 50C in a measuring device 1C in Modification Example 2 is configured to include only the first display unit 51C in which a plurality of strip-shaped display units 52C are arranged along Y axis direction from the first end portion 10a side toward the second end portion 10b side and the lengths in the X axis direction are set according to the widths of the case unit 10 on the “front surface” in the X axis direction at the arranged position with the X axis direction as the longitudinal direction. However, differently from Modification Example 1, each of the plurality of the strip-shaped display units 52C are arranged such that the widths in the Y axis direction become gradually larger (thicker) from the first end portion 10a side toward the second end portion 10b side.

According to the present modification example, it is possible to make the wearer visually recognize the value or degree of the displayed biological information with emphasizing the values or degrees.

### Modification Example 3

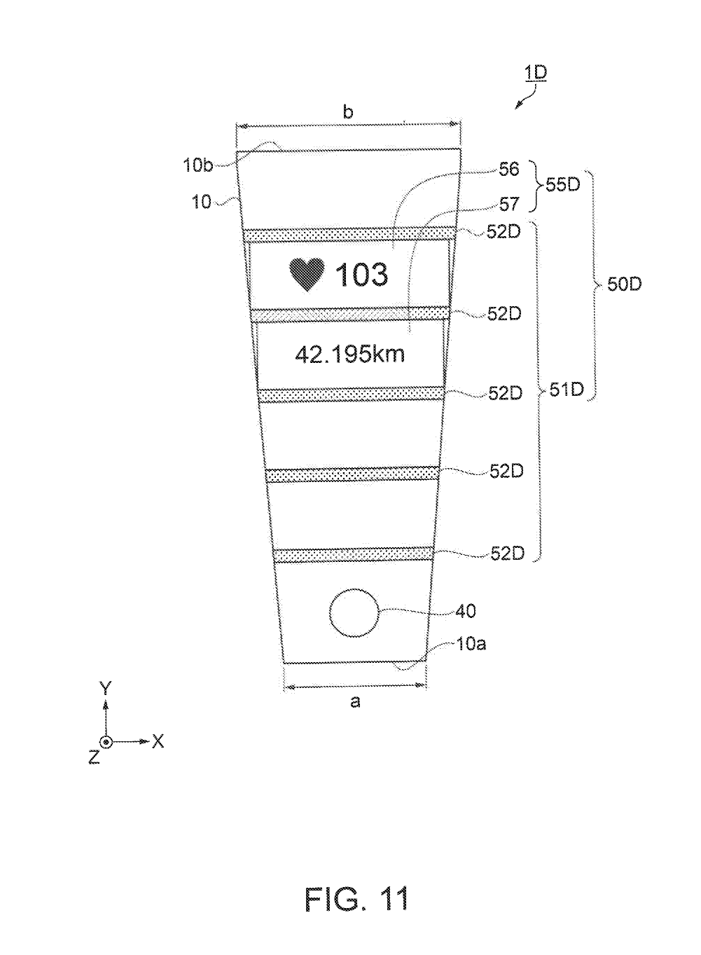

FIG. 11 is a plan view illustrating a schematic configuration of a display unit in the biological information measuring device in Modification Example 3.

In FIG. 11, as a configuration similar to that in Modification Example 1, the display unit 500 in a measuring device 10 in Modification Example 3 is configured to include the first display unit 510 in which a plurality of strip-shaped display units 520 are arranged along Y axis direction from the first end portion 10a side toward the second end portion 10b side and the lengths in the X axis direction are set according to the widths of the case unit 10 on the “front surface” in the X axis direction at the arranged position with the X axis direction as the longitudinal direction, and second display units 56 and 57 and the like such as LCDs and displaying the characters and images and arranged in the region between the adjacent strip-shaped display units 520 in the first display unit 51D.

According to this configuration, since the display (lighting) easy to visually understand by the number of strips of the strip-shaped display units 52D displayed on the first display unit 51D can be performed and the information can be displayed by the images such as the numeric value or the characters on the second display units 56 and 57, it is possible to notify the wearer of various information items in an easy-to-understand manner. For example, during an physical exercise such as running, information such as an elapsed time, level of physical exercise intensity, calories burned, the number of steps, a degree of fatigue, or other achievements against the goals set in advance can be roughly recognized by the number strips of the displayed (lighting) strip-shaped display units 52D of the first display unit 51D, and can be reflected on the physical exercise, and then, the specific measured values such as the physical exercise (running) distance and physical exercise time, the heart rate, the calorie consumption, the number of steps, and the degree of fatigue can be displayed on the second display units 56 and 57 as the characters and the images at a predetermined timing when the physical exercise is stopped. The number of second display units is not limited to two, and equal to more than two second display units may be arranged. In this way, it is possible to easily grasp various information items at a time. In addition, the number of strip-shaped display units 52D is not limited to five either, and three or seven strip-shaped display units 52D may be arranged.

### Modification Example 4

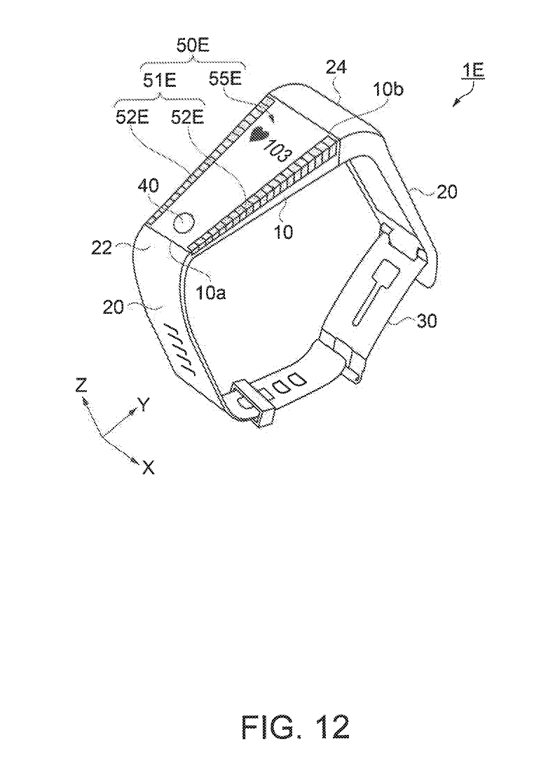

FIG. 12 is a perspective view illustrating a schematic configuration of a display unit in the biological information measuring device in Modification Example 4.

illustrated in FIG. 12, a display unit 50E of a measuring device 1E in Modification Example 4 includes a first display unit 51E having strip-shaped display units 52E at both end sides the “front surface” of the case unit 10 in the X axis direction arranged with the Y axis direction as the longitudinal direction. As described above, the strip-shaped display units 52E with the Y axis direction which is the longitudinal direction of the case unit 10 as the longitudinal direction become larger than the display units with the X axis direction as the longitudinal direction in Embodiment and Modification Examples 1 to 3 described above. Therefore, various information items can be displayed such that the wearer can easily understand. For example, it is possible to check the information such as an elapsed time, a level of physical exercise intensity, calories burned, the number of steps, a degree of fatigue, or other achievements against the goals set in advance at a glance by the length of displayed portion (lighting) of the strip-shaped display units 52E from the first end portion 10a side toward the second end portion 10b.

In addition, in the present modification example, the strip-shaped display units 52E are arranged at the corner portions of both sides of the case unit 10 in the X axis direction. In this way, the display on the strip-shaped display units 52E can be recognized from the “front surface” and the side surfaces of the case unit 10. Therefore, it is possible to obtain an effect that the visibility is improved.

In addition, in the present modification example, the strip-shaped display units 52 are provided in such a manner that the width of each strip-shaped display unit 52 in X axis direction of the “front surface” side and the width in the Z axis direction of the side surface side become larger from the first end portion 10a side toward second end portion 10b side. In this way, the visibility of the display on each strip-shaped display unit 52E can be improved and the level of or degree of the displayed information can be emphasized from the first end portion 10a side toward the second end portion 10b side. Therefore, it is possible to obtain an effect easily and intuitively grasping the situation.

In FIG. 12, the display unit 50E of the measuring device 1E in the present modification example includes a second display unit 55E configured to include LCDs displaying the information by the characters and images in addition to the first display unit 51E described above. In this way, it is possible to notify the user of the specific information such as the time and the measured value of the biological information. Therefore, the user can intuitively grasp the degrees of the strength of the physical exercise and the load to the body using the first display unit 51E, and can grasp the degrees of the strength of the physical exercise and the load to the body in detail by the numbers or characters, if necessary.

In the present modification example, the strip-shaped display units 52e of the first display unit 51E are described to be arranged at the corner portions at both sides of the case unit 10 in the X axis direction, but not limited thereto. For example, the strip-shaped display units 52E may be arrange only on the “front surfaces” at both sides of the case unit 10 in the X axis direction.

In addition, in the present modification example, the strip-shaped display units 52E are provided in such a manner that the widths of each strip-shaped display unit 52E in the X axis direction and in the Z axis direction become larger from the first end portion 10a side toward second end portion 10b side, but not limited thereto. The widths of the strip-shaped display units 52E the X axis direction and in Z axis direction may be the same.

In addition, the two strip-shaped display units 52E are arranged at both sides of the “front surface” of the case unit 10 in the X axis direction as in the present modification example, but not limited thereto. Only one strip-shaped display unit may be provided.

Furthermore, the display unit 50E in the present modification example is described to include a second display unit 55E displaying the information by the images in addition to the first display unit 51E including the strip-shaped display unit 52E described above. However, only the first display unit 51E having the above-described configuration may be included.

As above, the embodiments and modification examples of the biological information measuring device as the wearable device is described. However, those skilled in the art can easily understand that many modifications that do not practically depart from the new aspects and effects of the invention can be made. Therefore, all of those modification examples are assumed to be included within the cope of the invention. For example, the terms described in the specification and drawings at least once together with other terms having broader or same meaning can be replaced with that different terms at any position in the specification and the drawings. In addition, the configuration and operation of the biological information measuring unit, the biological information measuring device, and the like are not limited to those described in the present embodiments, and can be embodied in variously modifications.

For example, the operation unit 40 may not be provided in a region near the first end portion 10a on the “front surface” of the case unit as in the embodiments and modification examples described above, or may be arranged on the side surface of the case unit, not limited to the “front surface” By arranging like this, it is possible to secure a wider display region on the front surface, and passing the sleeves of a shirt can be improved. Furthermore, a touch sensor or an acceleration sensor that can detect a user's operation such as a tap without adopting the button can be provided as an operation unit. By adopting these sensors, the movable portion of the device can be decreased, and thus, it is possible to improve waterproofness.

In addition, in the shape of the front view (refer to FIG. 3) and the shape of the cross sectional view (refer to FIG. 4) of the case unit 10 Embodiment 1 and the shape of the front view (refer to FIG. 7) and shape of the cross sectional view (refer to FIG. 8) of the case unit 10A in Embodiment 2, the shape of the front view and the shape in cross sectional view may be a combination of exchanged shapes respectively. For example, it is possible to make the shape of the front view of the case unit to be the shape of the case unit 10 in Embodiment 1 illustrated in FIG. 3, and the shape of the cross sectional view of the case unit is the shape case unit 10A in Embodiment 2 illustrated in FIG. 8.

In addition, both the shapes of the front view and the cross sectional view of the case unit in the embodiments and modification examples described above are illustrated as shapes of a combination of straight lines. However, at least a part of the shapes may be a curve (acc shape). Particularly, by forming the surface of the case unit that comes in contact with the user's skin as a curved surface that follows the shape of the contact portion on the wrist where the measuring device (wearable device) is mounted, it is possible to improve the feeling of wearing.

In addition, a plurality of strip-shaped display units included in the display unit (the first display unit) in the embodiments and modification examples described above may not be arranged in a regular interval in the Y axis direction as illustrated in FIG. 3, FIG. 7, and FIG. 9 to FIG. 11, and may be arranged in changed intervals, than then, it is possible to notify the user of the displayed content in an emphasized manner.

What is claimed is:

1. A wearable device comprising:
a case unit;
a first band that is a member connected to a first end portion of the case unit for mounting the case unit on a body of a user; and
a second band that is a member connected to a second end portion of the case unit for mounting the case unit on the body of the user,
wherein, when a direction from the first band toward the second band is Y axis, a direction orthogonal to the Y axis in a plan view seen from a thickness direction of the case unit is X axis, and a direction orthogonal to the X axis and Y axis is Z axis, and when a width of the first end portion in the X axis direction is a and a width of the second end portion in the X axis direction is b, the case unit satisfies a relation a<b.

2. The wearable device according to claim 1,
wherein a ratio between a and b is a:b=1:(equal to or greater than 1.1 and equal to or smaller than 1.4).

3. The wearable device according to claim 1,
wherein, when a width of the first end portion in the Z axis direction is c and a width of the second end portion in the Z axis direction is d, the relation is c<d.

4. The wearable device according to claim 3,
wherein a ratio between c and d is c:d=1:(equal to greater than 1.2 to equal to or less than 4.0).

5. The wearable device according to claim 1,
wherein a middle portion having a width b′ in the X axis direction which is different from at least one of the a and the b is included between the first end portion and the second end portion in a plan view seen from the Z axis direction.

6. The wearable device according to claim 5,
wherein the relation between the a and the b′ is b′>a.

7. The wearable device according to claim 6,
wherein the relation between the b and the b′ is a≅b′ and b′≧b.

8. The wearable device according to claim 5,
wherein, when a width of the middle portion in the Z axis direction in a plan view seen from the X axis direction is d′, the relation is d′>c.

9. The wearable device according to claim 5,
wherein, when the width of the middle portion in the Z axis direction in a plan view seen from the X axis direction is d′, the relation is d′≧d.

10. The wearable device according to claim 1,
wherein the case unit includes a biological information measuring unit that measures biological information of the user.

11. The wearable device according to claim 1,
wherein a battery is included in the case unit, and
wherein a distance from the second end portion to the battery is shorter than a distance from the first end portion to the battery in a cross sectional view seen from the X axis direction.

12. The wearable device according to claim 1,
wherein an operation unit arranged on the case unit at the first end portion side in a plan view seen from the Z axis direction is included.

13. The wearable device according to claim 1,
wherein the case unit includes a first display unit on which a plurality of strip-shaped display units with the X axis direction as the longitudinal direction are arranged from the first end portion side toward the second end portion side.

14. The wearable device according to claim 13,
wherein widths of the strip-shaped display units in the Y axis direction become larger from the first end portion side toward the second end portion side.

15. The wearable device according to claim 13,
wherein a second display unit that displays an image is arranged in a region between the strip-shaped display units adjacent to each other.
